(19) United States
(12) Patent Application Publication
(10) Pub. No.: US 2020/0317616 A1
(43) Pub. Date: Oct. 8, 2020

(54) Crystalline Form of Betrixaban Maleate

(71) Applicant: Apotex Inc., Toronto (CA)

(72) Inventors: Fabio E. S. Souza, Mississauga (CA); Alexei Iarov, Richmond Hill (CA); Probal Kanti Datta, Hamilton (CA)

(21) Appl. No.: 16/835,377

(22) Filed: Mar. 31, 2020

Related U.S. Application Data
(60) Provisional application No. 62/828,593, filed on Apr. 3, 2019.

(51) Int. Cl. C07D 213/16

(52) U.S. Cl. CPC C07D 213/16; A61K 9/48; C07B 2200/13

(57) ABSTRACT

The present invention provides a novel crystalline form of Betrixaban maleate, Betrixaban maleate Form APO-I, including Betrixaban maleate and dimethyl sulfoxide, compositions and processes for the preparation thereof, and the use of this crystalline form in the treatment of conditions characterized by undesired thrombosis, and in particular, venous thromboembolism (VTE).

### CROSS-REFERENCE TO RELATED APPLICATION

This application claims the benefit of U.S. Provisional Patent Application No. 62/828,593, filed Apr. 3, 2019, the disclosure of which is hereby incorporated in its entirety by reference.

### TECHNICAL FIELD

The present invention is directed to novel crystalline forms of Betrixaban maleate, pharmaceutical compositions containing these forms, processes for their preparation, and their use in the treatment or prevention of a condition characterized by undesired thrombosis, including venous thromboembolism (VTE).

### BACKGROUND

Betrixaban (1), or N-(5-chloropyridin-2-yl)-2-[4-(N,N-dimethylcarbamimidoyl)-benzoylamino]-5-methoxybenzamide, in the form of a maleate salt, is the active ingredient in BEVYXXA®, which is indicated for the prophylaxis of venous thromboembolism (VTE) in adult patients hospitalized for an acute medical illness who are at risk for thromboembolic complications due to moderate or severe restricted mobility and other risk factors for VTE.

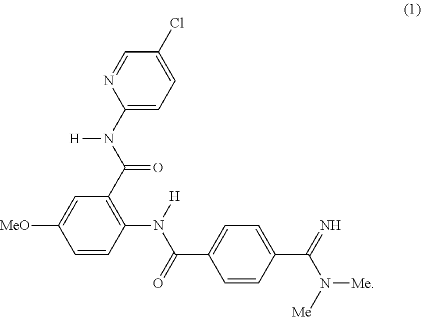 [SMILES: COc1ccc(NC(=O)c2ccc(C(=N)N(C)C)cc2)c(C(=O)Nc2ccc(Cl)cn2)c1]

Crystalline forms of Betrixaban maleate, including hydrated and solvated forms thereof, are reported in, for example, WO 2007/056517 A2, WO 2012/031017 A1, WO 2017/208169 A1 and WO 2018/069936 A1.

According to the review published by the U.S. Center for Drug Evaluation and Research (CDER) in connection with the approval of BEVYXXA® (NDA 208383), the drug substance Betrixaban maleate, which by reference to the chemical formula in the report is in the anhydrous form, has high solubility but low permeability, placing it in Class III of the Biopharmaceutics Classification System (BCS). Despite high solubility, for example, the labelling of BEVYXXA® indicates that the oral bioavailability of Betrixaban is only 34%.

Different crystalline forms of the same compound may have different crystal packing, thermodynamic, spectroscopic, kinetic, surface and mechanical properties. For example, different crystalline forms may have different stability properties such that a particular crystalline form may be less sensitive to heat, relative humidity (RH) and/or light. Different crystalline forms of a compound may also be more susceptible to moisture uptake, resulting in a potential alteration of physical characteristics of the form such as flowability, density or compressibility, which can lead to problems during formulation/tabletting and/or to changes in dissolution rate of the formulated drug product.

For example, unintended absorption of moisture by a hygroscopic crystalline form of a drug substance can alter its compressibility during tabletting, resulting in a softer tablet having a faster dissolution rate following administration. A particular crystalline form may provide more favourable compressibility and/or density properties, thereby providing more desirable characteristics for formulation and/or product manufacturing. Differences in stability between solid forms of a drug may result from changes in chemical reactivity, such as differential oxidation. Such properties may provide for more suitable product qualities, including a dosage form that is more resistant to discolouration when comprised of a specific crystalline form. Particular crystalline forms may also have different solubilities, thereby providing different pharmacokinetic parameters, which allow for specific crystalline forms to be used in order to achieve specific pharmacokinetic targets. Crystalline forms which incorporate a co-former molecule such as solvates may be imparted with properties arising from novel interactions between the compound and the co-former such as differences in permeability or solubility. Differences in permeability between crystalline forms are particularly relevant for compounds exhibiting low permeability, such as BCS Class III drug substances, where even a modest increase in permeability can provide a beneficial enhancement in bioavailability.

Although general approaches to crystalline form screening of active pharmaceutical ingredients are known, it is well established that the prediction of whether any given compound will exhibit polymorphism is not possible. Accordingly, it is not possible to extend generalities to the number and kinds of crystalline forms that can exist for Betrixaban maleate, or to what methods will be suitable for the preparation of any given crystalline form. Furthermore, prediction of the properties of any unknown crystalline forms, and how they will differ from other crystalline forms of the same compound, remains elusive (Joel Bernstein, Polymorphism in Molecular Crystals, Oxford University Press, New York, 2002, page 9).

Owing to the reported low bioavailability of Betrixaban maleate, there exists a need for novel crystalline forms of Betrixaban maleate having improved properties for use in providing drug products containing Betrixaban maleate, and commercially amenable processes for their manufacture.

### SUMMARY OF THE INVENTION

The Betrixaban maleate crystalline form of the present invention comprises Betrixaban maleate that has crystallized with dimethyl sulfoxide in the same crystal lattice. Dimethyl sulfoxide has an established safety record and can therefore safely be used in materials intended for use in the preparation of pharmaceutical compositions for administration to humans or animals. Further, dimethyl sulfoxide has been known to act as a permeability enhancing agent for some compounds. Thus, the provision of a crystalline form of Betrixaban maleate comprising dimethyl sulfoxide in the same crystal lattice is expected to provide improvements in the permeability of Betrixaban maleate.

The present invention provides a crystalline form of Betrixaban maleate that can be prepared by an efficient and industrially compatible process. Surprisingly, conditions have been discovered which afford the novel crystalline form of the present invention, rather than the known Form M1 that is reported to form from a solvent system comprising dimethyl sulfoxide.

Accordingly, in a first aspect of the present invention, there is provided a crystalline form of Betrixaban maleate comprising Betrixaban maleate and dimethyl sulfoxide. Preferably, in the crystalline form of the first aspect, the molar ratio of Betrixaban maleate to dimethyl sulfoxide is between approximately 1:0.75 and approximately 1:1.25. More preferably, the molar ratio of Betrixaban maleate to dimethyl sulfoxide in the crystalline form of the first aspect is approximately 1:1.

In a second aspect of the present invention, there is provided a crystalline form of Betrixaban maleate, APO-I, comprising Betrixaban maleate and dimethyl sulfoxide, characterized by a PXRD diffractogram comprising peaks, expressed in degrees 28 (±0.2°), at 8.3°, 13.3° and 14.9°. In a preferred embodiment of the second aspect, the PXRD diffractogram further comprises at least three peaks, expressed in degrees 28 (±0.2°), selected from the group consisting of: 4.2°, 12.2°, 14.5°, 16.8°, 17.4°, 18.2°, 18.6°, 19.0°, 21.7° and 26.0°. In a further preferred embodiment of the second aspect, the PXRD diffractogram further comprises peaks, expressed in degrees 28 (±0.2°), at 4.2°, 12.2°, 14.5°, 16.8°, 17.4°, 18.2°, 18.6°, 19.0°, 21.7° and 26.0°. Preferably, the crystalline form of the second aspect of the invention provides a PXRD diffractogram comprising peaks in substantially the same positions (±0.2° 28) as those shown in FIG. 1. In a further preferred embodiment of the second aspect, the molar ratio of Betrixaban maleate to dimethyl sulfoxide in the crystalline form is between approximately 1:0.75 and approximately 1:1.25.

In a third aspect of the present invention, there is provided a process for the preparation of a crystalline form of Betrixaban maleate according to the first or second aspects of the invention, the process comprising:
(1) Preparing a solution of Betrixaban maleate in dimethyl sulfoxide at a suitable temperature;
(2) Adding an organic anti-solvent to the solution to form a mixture;
(3) Optionally, seeding the mixture with seeds comprising a dimethyl sulfoxide solvate of Betrixaban maleate characterized by a PXRD diffractogram comprising peaks, expressed in degrees 2θ (±0.2°), at 8.3°, 13.3° and 14.9°;
(4) Cooling the mixture, if necessary, to form a suspension comprising Betrixaban maleate crystalline form containing dimethyl sulfoxide; and
(5) Isolating the Betrixaban maleate crystals from the suspension.

Preferably, in the third aspect of the present invention, there is provided a process for the preparation of a crystalline form of Betrixaban maleate according to the second aspect of the invention.

In a preferred embodiment of the third aspect, preparing a solution of Betrixaban maleate comprises dissolving Betrixaban maleate in dimethyl sulfoxide, preferably at a temperature between approximately 50° C. and approximately 70° C. In another preferred embodiment of the third aspect, preparing a solution of Betrixaban maleate comprises dissolving Betrixaban free base and maleic acid in dimethyl sulfoxide, preferably at a temperature between approximately 20° C. and approximately 30° C. In a further preferred embodiment of the third aspect, the organic anti-solvent is an alkyl ester, preferably an alkyl acetate. More preferably, the organic anti-solvent is an alkyl acetate wherein the alkyl portion has 1 to 5 carbon atoms. Most preferably the organic anti-solvent is ethyl acetate. In another preferred embodiment of the third aspect of the invention, the mixture is seeded. In another preferred embodiment of the third aspect, the molar ratio of Betrixaban maleate to dimethyl sulfoxide in the crystalline form prepared is between approximately 1:0.75 and approximately 1:1.25. Most preferably, the molar ratio of Betrixaban maleate to dimethyl sulfoxide in the crystalline form is approximately 1:1.

In a fourth aspect of the present invention, there is provided a pharmaceutical composition comprising a crystalline form of Betrixaban maleate according to the first or second aspects of the invention, or Betrixaban maleate prepared according to the process of the third aspect of the invention, and one or more pharmaceutically acceptable excipients. Preferably, the pharmaceutical composition is in the form of a solid dosage form. Most preferably, the pharmaceutical composition is a capsule. Preferably, the pharmaceutical composition of the fourth aspect comprises an amount of the crystalline form of Betrixaban maleate of the first or second aspects that is equivalent to 40 mg or 80 mg Betrixaban free base.

In a fifth aspect of the present invention, there is provided the use of a crystalline form of Betrixaban maleate according to the first or second aspects of the invention, the Betrixaban maleate prepared according to the process of the third aspect of the invention, or the pharmaceutical compositions of the fourth aspect of the invention, in the treatment or prevention of a condition characterized by undesired thrombosis. In a preferred embodiment of the fifth aspect, the condition associated with undesired thrombosis is venous thromboembolism (VTE).

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention are described, by way of example only, with reference to the attached Figure.

FIG. 1 is a representative PXRD diffractogram of Betrixaban maleate Form APO-I as prepared in Example 4.

### DETAILED DESCRIPTION

The Betrixaban maleate crystalline form of the present invention comprises Betrixaban maleate that has crystallized with dimethyl sulfoxide. Importantly, with respect to the use of this crystalline form in the preparation of pharmaceutical compositions, dimethyl sulfoxide is included in both the U.S. Food & Drug Administration's (FDA's) Substances Added to Food inventory (formerly Everything Added to Food in the United States (EAFUS)), and the Inactive Ingredient Database (IID). The EAFUS list contains ingredients added directly to food that the FDA has either approved as food additives, or has listed or affirmed as being GRAS (Generally Recognized As Safe). The IID list provides information on inactive ingredients present in FDA-approved drug products. Once an inactive ingredient has appeared in an approved drug product for a particular route of administration, the inactive ingredient is not considered new, and may require a less extensive review the next time it is included in a new drug product.

Furthermore, a number of regulated products for medical use comprise dimethyl sulfoxide as a component, either as part of the formulation or as part of the active ingredient itself. For example, RIMSO-50® is an aqueous dimethyl sulfoxide solution for intravesical instillation approved by the U.S. FDA for use in the symptomatic relief of interstitial cystitis. A dimethyl sulfoxide solvate of Trametinib is the active ingredient in the U.S. FDA approved drug product MEKINIST®, which is indicated for the treatment of certain types of melanoma.

Also, of importance to the present invention is that dimethyl sulfoxide is known to act as a permeability enhancer. Thus, the provision of a crystalline form of Betrixaban maleate comprising Betrixaban maleate and dimethyl sulfoxide is expected to provide improvements in the permeability of Betrixaban maleate, which has been classified according to the BCS as having poor permeability.

The Betrixaban maleate crystalline form of the present invention exhibits differences in properties when compared to the known crystalline forms of Betrixaban maleate. Properties that differ between the invention and known crystalline forms of Betrixaban maleate include crystal packing properties such as molar volume, density and hygroscopicity; thermodynamic properties such as melting point and solubility; kinetic properties such as dissolution rate and chemical/polymorphic stability; surface properties such as crystal habit/particle morphology; and/or mechanical properties such as hardness, tensile strength, compactibility, tabletting, handling, flow, and blending.

Further, the present invention provides a crystalline form of Betrixaban maleate that can be prepared by an efficient and industrially compatible process. Surprisingly, despite reports that another crystalline form, Form M1, is prepared from a solvent system comprising dimethyl sulfoxide and water, the crystalline form of the present invention, which incorporates dimethyl sulfoxide into its crystal lattice, can be prepared from a solvent mixture comprising dimethyl sulfoxide and an organic anti-solvent. Importantly, in addition to dimethyl sulfoxide, the preparation of the crystalline form of the present invention uses Class 3 solvents established by the ICH (International Council for Harmonisation of Technical Requirements for Pharmaceuticals for Human Use) as having low toxicity.

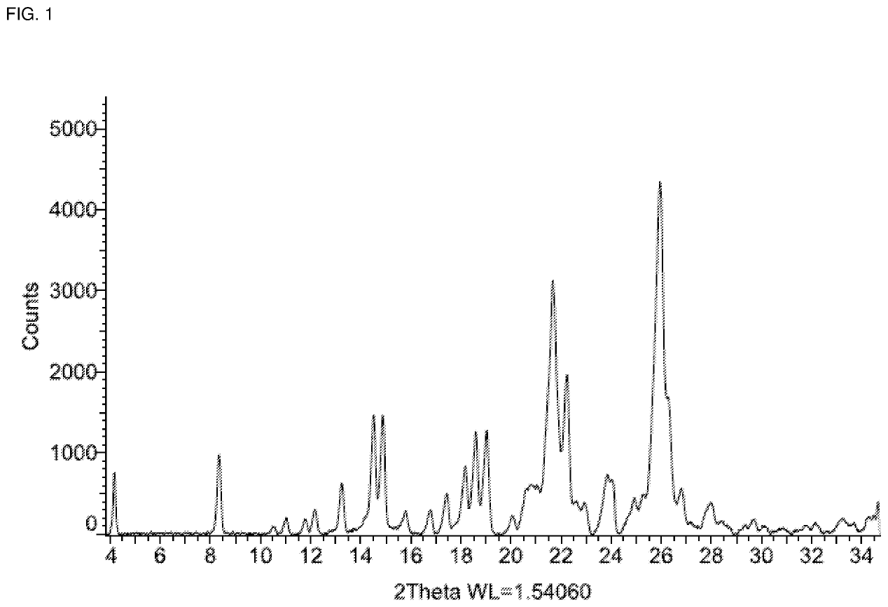

Depending on the manner in which the crystalline forms of the present invention are prepared, and the methodology and instrument used for PXRD analysis, the intensity of a given peak observed in a PXRD diffractogram of the crystalline form may vary when compared to the same peak in the representative PXRD diffractogram provided in FIG. 1. Thus, differences in relative peak intensities between peaks in a PXRD diffractogram for a given crystalline form may be observed when compared to the relative peak intensities of the peaks in the representative PXRD diffractogram of FIG. 1. Any such differences may be due, in part, to the preferred orientation of the sample and its deviation from the ideal random sample orientation, the preparation of the sample for analysis, and the methodology applied for the analysis. Such variations are known and understood by a person of skill in the art, and any such variations do not depart from the invention disclosed herein.

In addition to the differences in relative peak intensities that may be observed in comparison to the representative PXRD diffractogram provided in FIG. 1, it is understood that individual peak positions may vary between ±0.2° 2θ from the values observed in the representative PXRD diffractograms provided in FIG. 1 for the crystalline form of the invention, or listed in Table 1. Such variations are known and understood by a person of skill in the art, and any such variations do not depart from the invention disclosed herein.

Further, depending on the instrument used for X-ray analysis and its calibration, uniform offsets in the peak position of each peak in a PXRD diffractogram of greater that 0.2° 2θ may be observed when compared to the representative PXRD diffractogram provided in FIG. 1. Thus, PXRD diffractograms of the crystalline form of the present invention may, in some circumstances, display the same relative peak positions as observed in the representative PXRD diffractogram provided in FIG. 1, with the exception that each peak is offset in the same direction, and by approximately the same amount, such that the overall PXRD diffractogram is substantially the same in appearance as the PXRD diffractogram of FIG. 1, with the exception of the uniform offset in peak positions. The observation of any such uniform peak shift in a PXRD diffractogram does not depart from the invention disclosed herein given that the relative peak positions of the individual peaks within the PXRD diffractogram remain consistent with the relative peak positions observed in the PXRD diffractogram of FIG. 1.

As used herein, the term ‘crystalline form’ refers to a substance with a particular arrangement of molecular components in its crystal lattice, and which may be identified by physical characterization methods such as PXRD. As used herein, the term crystalline form is intended to include single-component and multiple-component crystalline forms. Single-component forms of Betrixaban maleate, such as those known in the art, consist solely of Betrixaban maleate in the repeating unit of the crystal lattice. Multiple-component forms of Betrixaban maleate, such as those of the present invention, include crystalline forms of Betrixaban maleate wherein one or more other molecules are also incorporated into the crystal lattice with Betrixaban maleate.

Multi-component crystalline forms comprising more than one type of molecule in the crystalline lattice may have some variability in the exact molar ratio of their components depending on the conditions used for their preparation. For example, a molar ratio of components within a multi-component crystalline form provides a person of skill in the art information as to the general relative quantities of the components of the crystalline form. In many cases, the molar ratio may vary by ±25% from a stated range. With respect to the present invention, a molar ratio of 1:1 should be understood to include the ratios 1:0.75 and 1:1.25, as well as all of the individual ratios in between.

As used herein, the term “room temperature” refers to a temperature in the range of 20° C. to 25° C.

When describing the embodiments of the present invention there may be a common variance to a given temperature or time that would be understood or expected by the person skilled in the art to provide substantially the same result. For example, when reference is made to a particular temperature, it is to be understood by the person skilled in the art that there is an allowable variance of ±5° C. associated with that temperature. When reference is made to a particular time, it is to be understood that there is an allowable variance of ±10 minutes when the time is one or two hours, and ±1 hour when longer periods of time are referenced.

In one embodiment of the present invention, there is provided a new crystalline form of Betrixaban maleate, Betrixaban maleate Form APO-I, comprising Betrixaban maleate and dimethyl sulfoxide. Preferably, in Betrixaban maleate Form APO-I, the molar ratio of Betrixaban maleate to dimethyl sulfoxide is approximately 1:1.

Betrixaban maleate Form APO-I can be characterized by a PXRD diffractogram comprising, among other peaks, characteristic peaks, expressed in degrees 2θ (±0.2°), at 8.3°, 13.3° and 14.9°. Preferably, the PXRD diffractogram further comprises at least three peaks, expressed in degrees 2θ (±0.2°), selected from the group consisting of 4.2°, 12.2°, 14.5°, 16.8°, 17.4°, 18.2°, 18.6°, 19.0°, 21.7° and 26.0°. More preferably, the PXRD diffractogram further comprises peaks, expressed in degrees 2θ (±0.2°), at 4.2°, 12.2°, 14.5°, 16.8°, 17.4°, 18.2°, 18.6°, 19.0°, 21.7° and 26.0°.

An illustrative PXRD diffractogram of Betrixaban maleate Form APO-I, as prepared in Example 4, is shown in FIG. 1. A peak listing, comprising representative peaks from the PXRD diffractogram in FIG. 1, and their relative intensities, is provided in Table 1. Although illustrative of the PXRD diffractogram that is provided for the Betrixaban maleate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 1
Relative peak intensities of Betrixaban
maleate Form APO-I from FIG. 1

| Angle (2θ) | Relative intensity (%) |
|---|---|
| 4.17 | 17.3 |
| 8.35 | 22.5 |
| 11.03 | 4.6 |
| 11.79 | 4.1 |
| 12.18 | 6.9 |
| 13.25 | 14.3 |
| 14.52 | 33.8 |
| 14.89 | 33.5 |
| 15.80 | 6.5 |
| 16.79 | 6.7 |
| 17.44 | 11.4 |
| 18.18 | 19.2 |
| 18.60 | 29.0 |
| 19.03 | 29.3 |
| 21.67 | 71.9 |
| 22.23 | 45.1 |
| 23.91 | 15.7 |
| 25.96 | 100.0 |

In a further embodiment of the invention, there is provided a process for the preparation of Betrixaban maleate Form APO-I, the process comprising:
(1) Preparing a solution of Betrixaban maleate in dimethyl sulfoxide at a suitable temperature;
(2) Adding an organic anti-solvent to the solution to form a mixture;
(3) Optionally, seeding the mixture, with seeds comprising a dimethyl sulfoxide solvate of Betrixaban maleate characterized by a PXRD diffractogram comprising peaks, expressed in degrees 2θ (±0.2°), at 8.3°, 13.3° and 14.9°;
(4) Cooling the mixture, if necessary, to form a suspension comprising Betrixaban maleate crystals containing dimethyl sulfoxide; and
(5) Isolating the Betrixaban maleate crystals from the suspension.

The step of preparing a solution of Betrixaban maleate in dimethyl sulfoxide may involve dissolving the Betrixaban maleate salt in dimethyl sulfoxide or it may involve dissolving Betrixaban free base and maleic acid in dimethyl sulfoxide to form Betrixaban maleate. Preferably, when Betrixaban maleate is used, the suitable temperature for dissolution is elevated, and is preferably between approximately 50° C. and approximately 70° C. When Betrixaban free base and maleic acid are used, the suitable temperature for dissolution is preferably between approximately 20° C. and approximately 30° C.

The organic anti-solvent may be an alkyl ester, and is preferably an alkyl acetate. More preferably, the organic anti-solvent is an alkyl acetate wherein the alkyl portion has 1 to 5 carbon atoms and is selected from the group consisting of methyl acetate, ethyl acetate, n-propyl acetate, i-propyl acetate and n-butyl acetate. Most preferably the organic anti-solvent is ethyl acetate.

The Betrixaban maleate solution may be seeded with Betrixaban maleate Form APO-I, either before, during or after addition of the organic anti-solvent. Preferably, seed crystals can, in the first instance, be prepared by conducting the process for preparation of Form APO-I using n-butyl acetate as the organic anti-solvent as described in Example 2. Thereafter, seed crystals for future preparations can also be reserved from Form APO-I prepared by any method, for example, as described in Examples 3 and 4.

Following addition of the anti-solvent and optional seeding, the resulting suspension can be cooled, preferably to 0-5° C. Filtration of the suspension and drying in vacuo under high vacuum, preferably at room temperature, affords Betrixaban maleate Form APO-I having a PXRD diffractogram consistent with FIG. 1.

In a further embodiment of the invention, there is provided a pharmaceutical composition of a crystalline form of Betrixaban maleate comprising Betrixaban maleate and dimethyl sulfoxide with one or more pharmaceutically acceptable excipients. Preferably, the pharmaceutical composition is a solid dosage form suitable for oral administration, such as a capsule, tablet, pill, powder or granulate. Most preferably, the pharmaceutical composition is a capsule. Preferably, the pharmaceutical composition provides a dose of Betrixaban maleate that is equivalent to the 40 mg or 80 mg of Betrixaban free base found in BEVYXXA® drug products.

Suitable pharmaceutically acceptable excipients are preferably inert with respect to the crystalline form of Betrixaban maleate of the present invention, and may include, for example, one or more excipients selected from binders such as lactose, starches, modified starches, sugars, gum acacia, gum tragacanth, guar gum, pectin, wax binders, microcrystalline cellulose, methylcellulose, carboxymethylcellulose, hydroxypropyl methylcellulose, hydroxyethyl cellulose, hydroxypropyl cellulose, copolyvidone, gelatine, polyvinylpyrrolidone (PVP) and sodium alginate; fillers or diluents such as lactose, sugar, starches, modified starches, mannitol, sorbitol, inorganic salts, cellulose derivatives (e.g., microcrystalline cellulose, cellulose), calcium sulphate, xylitol and lactitol; disintegrants such as croscarmellose sodium, crospovidone, polyvinylpyrrolidone, sodium starch glycollate, corn starch, microcrystalline cellulose, hydroxypropyl methylcellulose and hydroxypropyl cellulose; lubricants such as magnesium stearate, magnesium lauryl stearate, sodium stearyl fumarate, stearic acid, calcium stearate, zinc stearate, potassium benzoate, sodium benzoate, myristic acid, palmitic acid, mineral oil, hydrogenated castor oil, medium-chain triglycerides, poloxamer, polyethylene glycol and talc; and dispersants or solubility enhancing agents, such cyclodextrins, glyceryl monostearate, hypromellose, meglumine, Poloxamer, polyoxyethylene castor oil derivatives, polyoxyethylene stearates, polyoxylglycerides, povidone, and stearic acid. Other excipients including preservatives, stabilisers, anti-oxidants, silica flow conditioners, antiadherents or glidants may be added as required. Other suitable excipients and the preparation of solid oral dosage forms is well known to person of skill in the art, and is described generally, for example, in Remington The Science and Practice of Pharmacy 21^{st} Edition (Lippincott Williams & Wilkins: Philadelphia; 2006; Chapter 45).

Optionally, when the pharmaceutical compositions are solid dosage forms, the solid dosage forms may be prepared with coatings, such as enteric coatings and extended release coatings, using standard pharmaceutical coatings. Such coatings, and their application, are well known to persons skilled in the art, and are described, for example, in Remington The Science and Practice of Pharmacy 21^{st} Edition (Lippincott Williams & Wilkins: Philadelphia; 2006; Chapter 47).

### EXAMPLES

The following non-limiting examples are illustrative of some of the aspects and embodiments of the invention described herein.

The Betrixaban maleate used as a starting material in the following example was consistent with Form I Betrixaban maleate which is reported in WO 2007/056517 A1. However, other polymorphic forms are equally suitable as starting material, provided dissolution of the form occurs when preparing the novel crystalline form of Betrixaban maleate of the present invention.

### PXRD Analysis:

PXRD diffractograms were recorded on a Bruker D8 Discover powder X-ray diffractometer (Bruker-AXS, Karlsruhe, Germany). The generator was a Micro-focus X-ray source (IMSTube: Cu tube with 1.54060 Å) with a voltage of 50 kV and current of 1.00 mA, using a divergence slit of 0.3 mm and collimator of 0.3 mm. For each sample, one frame was collected using a still scan with a Pilatus 3R-100 kA detector at the distance of 154.72 mm from the sample. Raw data were evaluated using the program EVA (Bruker-AXS, Karlsruhe, Germany).

### Example 1: Preparation of betrixaban maleate Form APO-I

Betrixaban free base (200 mg) and maleic acid (62 mg) were dissolved in dimethyl sulfoxide (0.6 mL) at room temperature. To this solution was added ethyl acetate (3.0 mL) and seeds of material prepared in Example 2 (ca. 5 mg) in one portion. The resulting suspension was stirred at room temperature for 3 hours, after which the solids were collected by vacuum filtration, washed with ethyl acetate (2×1 mL), and dried in vacuo at room temperature for 16 hours to afford Betrixaban maleate Form APO-I as a white solid (124 mg) having a PXRD diffractogram consistent with FIG. 1.

### Example 2: Preparation of Seeds for Use in the Preparation of betrixaban maleate Form APO-I

Betrixaban free base (200 mg) and maleic acid (61 mg) were dissolved in dimethyl sulfoxide (0.6 mL) at room temperature. To this solution was added n-butyl acetate (3.0 mL) in one portion. The resulting suspension was stirred at room temperature for 3 hours, after which the solids were collected by vacuum filtration, washed with ethyl acetate (2×1 mL) and dried in vacuo at room temperature for 16 hours to afford Betrixaban maleate Form APO-I as a white solid (174 mg) having a PXRD diffractogram consistent with FIG. 1.

### Example 3: Preparation of betrixaban maleate Form APO-I

Betrixaban free base (200 mg) and maleic acid (62 mg) were dissolved in dimethyl sulfoxide (0.6 mL) at room temperature. To this solution was added isopropyl acetate (3.0 mL) and seeds of material prepared in Example 1 (ca. 5 mg) in one portion. The resulting suspension was stirred at room temperature for 3 hours, after which the solids were collected by vacuum filtration, washed with ethyl acetate (2×1 mL), and dried in vacuo at room temperature for 16 hours to afford Betrixaban maleate Form APO-I as a white solid (178 mg) having a PXRD diffractogram consistent with FIG. 1.

### Example 4: Preparation of betrixaban maleate Form APO-I

Betrixaban maleate (3.80 g) was dissolved in dimethyl sulfoxide (16.2 g) at 60° C. The resulting solution was allowed to cool to room temperature, after which ethyl acetate (80 mL) and seeds of material prepared in Example 2 (ca. 20 mg) were added in one portion. The resulting suspension was stirred at room temperature for 16 hours and then cooled in an ice bath for 1 hour. The precipitated solids were collected by vacuum filtration, washed with ethyl acetate (2×5 mL), and dried in vacuo at room temperature for 16 hours to afford Betrixaban maleate Form APO-I as a white solid (3.60 g). The PXRD diffractogram of a sample prepared by this method is shown in FIG. 1. ¹H NMR analysis of the solid (DMSO-d6, 300 MHz) indicated a molar ratio of Betrixaban maleate:DMSO of approximately 1:1.

¹H-NMR of Betrixaban maleate Form APO-I (DMSO-d₆, 300 MHz) δ: 2.54 (s, 6H), 3.35 (br s, 6H), 3.86 (s, 3H), 6.02 (s, 2H), 7.20 (dd, J=2.9, 9.0 Hz, 1H), 7.44 (d, J=2.9 Hz, 1H), 7.76 (d, J=8.3 Hz, 2H), 7.96 (dd, J=2.6, 8.9 Hz, 1H), 8.01 (d, J=8.9 Hz, 1H), 8.05-8.15 (m, 3H), 8.45 (d, J=2.5 Hz, 1H), 9.20 (v br s), 11.06 (v br s).

What is claimed is:

1. A crystalline form of Betrixaban maleate comprising Betrixaban maleate and dimethyl sulfoxide.

2. The crystalline form of claim 1, wherein the molar ratio of Betrixaban maleate to dimethyl sulfoxide is between approximately 1:0.75 and approximately 1:1.25.

3. The crystalline form of claim 1, wherein the molar ratio of Betrixaban maleate to dimethyl sulfoxide is approximately 1:1.

4. A crystalline form of Betrixaban maleate comprising Betrixaban maleate and dimethyl sulfoxide characterized by a PXRD diffractogram comprising peaks, expressed in degrees 2θ (±0.2°), at 8.3°, 13.3° and 14.9°.

5. The crystalline form of claim 4, further comprising at least three peaks, expressed in degrees 2θ (±0.2°), selected from the group consisting of: 4.2°, 12.2°, 14.5°, 16.8°, 17.4°, 18.2°, 18.6°, 19.0°, 21.7° and 26.0°.

6. The crystalline form of claim 4, further comprising peaks, expressed in degrees 2θ (±0.2°), at 4.2°, 12.2°, 14.5°, 16.8°, 17.4°, 18.2°, 18.6°, 19.0°, 21.7° and 26.0°.

7. The crystalline form of claim 4 providing a PXRD diffractogram comprising peaks in substantially the same positions (±0.2° 2θ) as those shown in FIG. 1.

8. The crystalline form of claim 4, wherein the molar ratio of Betrixaban maleate to dimethyl sulfoxide is between approximately 1:0.75 and approximately 1:1.25.

9. The crystalline form of claim 8, wherein the molar ratio of Betrixaban maleate to dimethyl sulfoxide is approximately 1:1.

10. A process for the preparation of the crystalline form of Betrixaban maleate of claim 4, the process comprising:
(1) Preparing a solution of Betrixaban maleate in dimethyl sulfoxide at a suitable temperature;
(2) Adding an organic anti-solvent to the solution to form a mixture;
(3) Optionally, seeding the mixture, with seeds comprising a dimethyl sulfoxide solvate of Betrixaban maleate characterized by a PXRD diffractogram comprising peaks, expressed in degrees 2θ (±0.2°), at 8.3°, 13.3° and 14.9°;
(4) Cooling the mixture, if necessary, to form a suspension comprising Betrixaban maleate crystals containing dimethyl sulfoxide; and
(5) Isolating the Betrixaban maleate crystals form from the suspension.

11. The process of claim 10, wherein preparing the solution of Betrixaban maleate comprises dissolving Betrixaban maleate in dimethyl sulfoxide.

12. The process of claim 11, wherein the suitable temperature is between approximately 50° C. and approximately 70° C.

13. The process of claim 10, wherein preparing the solution of Betrixaban maleate comprises dissolving Betrixaban free base and maleic acid in dimethyl sulfoxide.

14. The process of claim 10, wherein the organic anti-solvent is an alkyl ester.

15. The process of claim 14, wherein the alkyl ester is an alkyl acetate wherein the alkyl portion has 1 to 5 carbon atoms.

16. The process of claim 10, wherein the solution is seeded.

17. A pharmaceutical composition comprising the crystalline form of Betrixaban maleate according to claim 4, and one or more pharmaceutically acceptable excipients.

18. The pharmaceutical composition of claim 17, wherein the pharmaceutical composition is a capsule.

19. A method of treating or preventing a condition characterized by undesired thrombosis comprising administering to a patient an effective amount of the crystalline form according to claim 4.

20. The method of 19, wherein the condition characterized by undesired thrombosis is venous thromboembolism (VTE).
